(19) United States
(12) Patent Application Publication
(10) Pub. No.: US 2004/0077974 A1
(43) Pub. Date: Apr. 22, 2004

(54) Laparoscopic device for sound detection

(76) Inventor: Mark Moore, Tallahassee, FL (US)

Correspondence Address: Carnes, Cona & Dixon, Innovation Park, 1673 West Paul Dirac Drive, Tallahassee, FL, 32310-3763, US

(21) Appl. No.: 10/277,441

(22) Filed: Oct. 22, 2002

(51) Int. Cl. A61B 5/103

(52) U.S. Cl. 600/587

(57) ABSTRACT

A sound detection device useful in the performance of laparoscopic procedures on a body. The device includes an elongated hollow tube having dimensions capable of passage through standard sized surgical ports in the body, which ports are used during the laparoscopic procedures. A sound detector is disposed in a first end of the tube for detecting sounds produced by anatomic vessels within the body. A transmitter is disposed in the tube with an input coupled to an output of the sound detector for transmitting signals indicative of sounds produced by the anatomic vessels. The device also includes a receiver external to the tube, which receiver is tuned for receiving signals transmitted by the transmitter. Moreover, the device includes an amplifier with an input coupled to an output of the receiver for amplifying signals received from the receiver, and the device includes a speaker coupled to an output of the amplifier for converting the signals to sound waves, whereby a physician performing the laparoscopic procedure while listening to the sound waves may decipher anatomic vessels from other organs within the body.

### BACKGROUND OF THE INVENTION

1. Field of the Invention

The present invention relates generally to a laparoscopic device used in laparoscopic surgery and more particularly to a laparoscopic instrument designed and configured to transmit sound from the patient to a receiver system so as to enable the surgical team to hear anatomic vessel sounds during laparoscopic procedures so as to provide a means for identifying arterial vessel structures.

2. Description of the Prior Art

With the advance of technology, the use of laparoscopic operative procedures, whereby the surgeon can work on the inside of the body with instruments through tiny ports inserted through the skin, is replacing many traditional “open” surgeries. Laparoscopy is used to diagnose and treat a range of abdominal or pevicle problems through a laparoscope that is placed in the abdomen through a small incision.

An advantage of laparoscopic operative procedures is that it is less traumatic to the patient and allows for quicker recovery time. In addition, laparoscopic procedures have less morbidity and mortality when compared to procedures performed with larger abdominal incisions. Generally, laparoscopy is performed under general anesthesia. The laparoscope is passed into the abdomen through an incision made in the belly button. The physician may also make one or more additional small incisions nearby in order to pass specialized surgical instruments into the abdomen. The abdomen is filled with carbon dioxide gas so as to separate the walls and provide the physician with a view.

Other viewing methods include the use of cameras. This will provide the physician with a means for viewing the surgical progression on a video monitor. When performing the laparoscopic operative procedure, although the surgeon is able to view the progression, he does not have a sense of touch or sound.

Conventionally, when operating on an “open” surgery, the surgeon has a sense of feel to identify certain structures, such as arteries. The surgeon can use his sense of feel and identify a pulsing structure as an artery, and thus avoid puncturing any major artery or vital organs. Without the sense of touch and sound, as with laparoscopic surgery, the surgeon may be unable to identify certain structures, thus impeding the laparoscopic operative procedure by limiting the surgeon's perception of the operating field.

Though several laparoscopic tools exist for aiding and assisting the surgeon when performing laparoscopic surgery, none specifically address the need for listening to sounds inside the body during an operation. For example, in U.S. Pat. No. 6,050,960 issued to Ferzli, there is disclosed a laparoscopic instrument designed and configured to render internal distance measurements within a body. In U.S. Pat. No. 6,086,606 issued to Knodel et al., there is disclosed a manually operated surgical tool that is ideally suited for laparoscopic surgeries.

As observed, a laparoscopic surgery is advantageous for a patient. The field is growing due to the reduced patient recovery time and to the increased rate of success performed by the surgeons. To further assist the surgeon, it would be advantageous to provide a device that is capable of transmitting internal sounds to the surgeon during an operation. Such a sound transmission device would be instrumental in providing successful surgical procedures, by rendering a device that will aid the surgical team in audibly distinguishing otherwise unidentifiable artery structures. Accordingly, it is seen that there is a need for a laparoscopic device designed and configured to provide sound transmission from the patient to the surgical team so as to offer the most advanced technology available today to the medical community.

As can be seen, these previous efforts do not provide a laparoscopic device that, when placed next to an internal structure, will wirelessly transmit any audible sound. The present invention will provide for audible sound to be transmitted by a system that utilizes conventional electronic circuitry, or IC chips, that are capable of handling any radio frequency. The received sounds will be transmitted to a receiver system that is located outside the patient during the operation. This arrangement will allow for the surgeon and surgical team to hear and listen to any and all sounds so as to provide a means for distinguishing unidentifiable arterial structures.

Consequently, the present invention provides a wireless laparoscopic device that features hands-free convenience and an enhanced diagnostic tool, by enabling wireless communication to the surgeon and surgical term during laparoscopic operative procedures, and one that innately provides a device that would be easily adaptable and successfully used by the medical community. Additionally, prior techniques do not suggest the present inventive combination of component elements as disclosed and claimed herein.

Thus, as will be seen, the present invention achieves its intended purposes, objectives and advantages over the prior art devices by accomplishing the needs and objectives as identified herein, through a new, useful and unobvious combination of component elements, which is simple to use, with the utilization of a minimum number of functioning parts, at a reasonable cost to manufacture, assemble, test and by employing only readily available material.

### SUMMARY OF THE INVENTION

The present invention is a novel and unique wireless laparoscopic device that will advance the state of the art of medicine by providing a higher level of patient care. This wireless laparoscopic device will greatly advance the current laparoscopic operative procedures by allowing the surgeon to listen to wirelessly transmitted anatomic vessel sounds during the operation/procedure. This novel and unique wireless laparoscopic device is designed to be compact in size and one that requires a minimal amount of components so as to adequately and successfully operate and manipulate the present invention.

To enable a wireless laparoscopic device, the present invention is comprised of an elongated insertable laparoscopic instrument having a sound detector device incorporated therein. The elongated laparoscopic instrument will appear to be conventional in exterior appearance, and thus will include a hollow tubular body having a first end and a second end. The hollow tubular body is made of dimensions that are capable of passage through standard sized ports used in conventional laparoscopic procedures.

Located in proximity to the first end of the hollow tubular body is a sound detection device. This detection device will provide a unit that will detect and transmit sound from the patient to an external receiver system. The receiver system is located outside the patient and will enable the detected sound to be amplified and heard via the receiver system. This will allow the surgeon or surgical team to constantly hear anatomic vessel sounds during laparoscopic procedures so as to provide a means to identify arterial vessel structures.

Using and operating the present invention is simple, easy, and extremely efficient. The present invention will elevate the role of laparoscopic operative procedures in modern medicine. In addition, the novel and unique laparoscopic device will greatly advance the technology in the art of laparoscopic operative procedures.

An alternate embodiment of the present invention comprises a laparoscopic instrument having a sound detector as described above, but includes an electrical conductor extending from a second end of the hollow tube out to an amplifier and sound reproducing speaker. This embodiment eliminates the need for a transmitter and receiver.

Accordingly, it is a primary object of the present invention to provide for a wireless laparoscopic device to be used by surgeons with the capability of enabling the surgical team to hear anatomic vessel sounds during laparoscopic procedures as a means to identify arterial vessel structures. Thus, a sound detection device useful in the performance of laparoscopic procedures on a body is provided. The device includes an elongated hollow tube having dimensions capable of passage through standard sized surgical ports in the body, which ports are used during the laparoscopic procedures. A sound detector is disposed in a first end of the tube for detecting sounds produced by anatomic vessels within the body. A transmitter is disposed in the tube with an input coupled to an output of the sound detector for transmitting signals indicative of sounds produced by the anatomic vessels. The device also includes a receiver external to the tube, which receiver is tuned for receiving signals transmitted by the transmitter. Moreover, the device includes an amplifier with an input coupled to an output of the receiver for amplifying signals received from the receiver, and the device includes a speaker coupled to an output of the amplifier for converting the signals to sound waves, whereby a physician performing the laparoscopic procedure while listening to the sound waves may decipher anatomic vessels from other organs within the body.

Another object of the present invention is to provide for a laparoscopic device that is compact in size and one that is adaptable for insertion into small surgical portals of a patient's body.

Still a further object of the present invention, to be specifically enumerated herein, is to provide a wireless device in accordance with the preceding objects and which will conform to conventional forms of manufacture, be of simple construction and easy to use so as to provide a device that would be economically feasible, long lasting and relatively trouble free in operation.

Although there have been laparoscopic tool devices developed and designed to advance and assist in laparoscopic operative procedures, none of the inventions address the need to listen to internal sounds within a body cavity during an operation. Thus, it is seen that the present invention provides a device that is instrumental in performing successful laparoscopic procedures. The product disclosed herein is sufficiently compact, low cost, and reliable enough to become commonly used by surgeons or surgical teams. The present invention is simplified in design, compact in size, and can be utilized easily, efficiently and successfully.

The foregoing has outlined some of the pertinent objects of the present invention. These objects should be construed to be merely illustrative of some of the more prominent features and application of the intended invention. Many other beneficial results can be obtained by applying the disclosed invention in a different manner or modifying the invention within the scope of the disclosure. Accordingly, a fuller understanding of the invention may be had by referring to the detailed description of the preferred embodiments in addition to the scope of the invention taken in conjunction with the accompanying drawings.

### BRIEF DESCRIPTION OF THE DRAWINGS

FIG. 1 is a side planar view of the components used in the laparoscopic device of the present invention, including the laparoscopic instrument and the receiver device.

FIG. 2 is a side view of the laparoscopic instrument used with the device of the present invention.

FIG. 3 is a cross-sectional view of the laparoscopic instrument used with the device of the present invention.

FIG. 4 is a block diagram, illustrating the electrical components utilized with the medical device of the present invention.

FIG. 5 is a side planar view of the components used in the laparoscopic device of the present invention, illustrating an alternative configuration for the laparoscopic instrument.

FIG. 6 is a cross-sectional view of the laparoscopic instrument used with the device of the present invention.

Similar reference numerals refer to similar parts throughout the several views of the drawings.

### DESCRIPTION OF THE PREFERRED EMBODIMENT

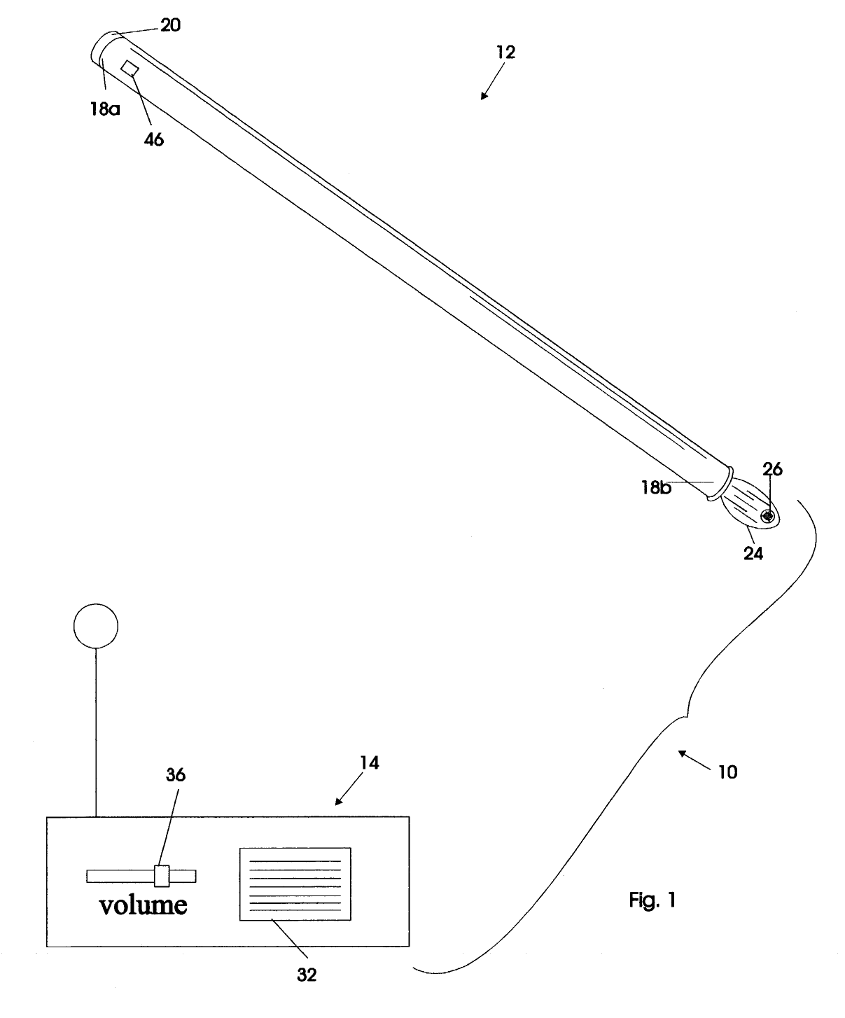

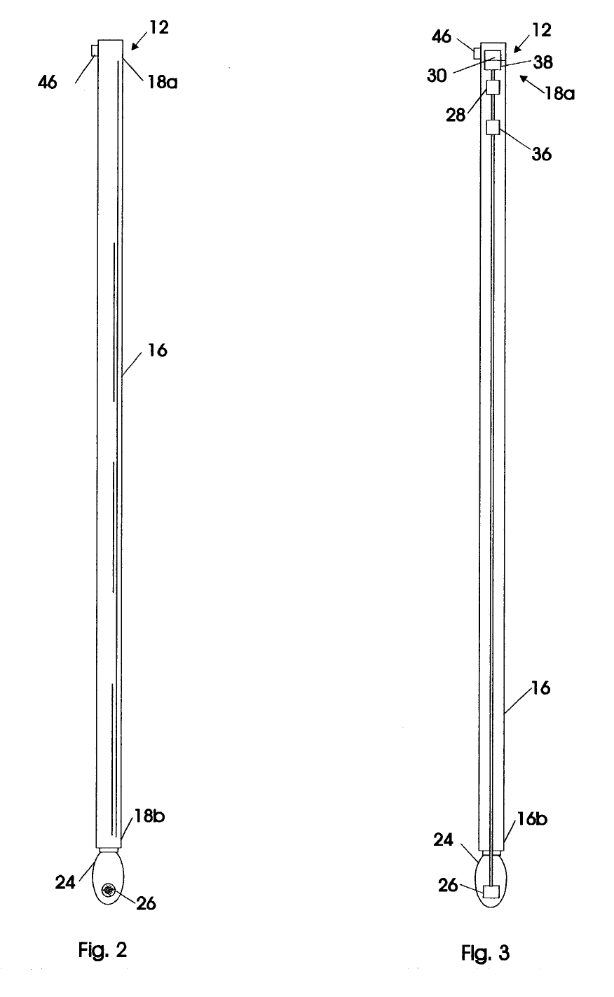

As seen in the drawings, and in particular with reference to FIGS. 1-3, thereof, the first embodiment of the present invention is shown. This is a novel and unique medical device for wireless laparoscopic procedures, denoted by reference numeral 10, which will be described herein. Shown is a wireless medical laparoscopic device 10, which comprises a conventional elongated insertable laparoscopic instrument 12 and a receiver system 14. The device of the present invention is designed and configured to detect anatomic vessel sounds during laparoscopic procedures so as to provide a means for identifying arterial vessel structures. Thus, the laparoscopic instrument senses and transmits the sound, while the receiver 14 amplifies the sound so as to be heard by the attending physician and the surgical team.

Thus, it is seen that in use the laparoscopic instrument 12 will be inserted into a patient and the anatomic vessel sounds detected by the instrument 12 will be transmitted to the receiver system 14.

The elongated laparoscopic instrument will appear to be conventional in exterior appearance, and thus will include a hollow tubular body 16. This hollow tubular body 16 includes a first end 18a and a second end 18b.

An end cap 20, as illustrated can be secured to the first end of the hollow tubular body. In the preferred embodiment, this end cap is removably secured thereto so as to provide access to the interior.

Secured to the second end 22 is a tip 24. This tip is hollow and is used to house a microphone 26. The microphone is protected via conventional means and thus includes a protective mesh covering. Coupled to the microphone is a transmitter 28. The transmitter is located in proximity to the first end of the hollow tubular body. The transmitter 28 will enable the received sounds from the microphone to be transmitted to the receiver system 14.

The microphone and transmitter form a transmitting system. To enable the transmitting system to work, power must be supplied thereto. The power supply in the preferred embodiment is supplied via a battery system 30 that is housed within the first end of the hollow tubular body 16. A switch 46 can be coupled between the power source and transmitting system so as to enable the unit to function when desired while still conserving energy. The power supply is stored within its respective housing so as to provide for the power supply to be replaced when necessary.

The receiving system is conventional and includes a means of receiving the signals from the transmitter 28 of the transmitting system. The received signals are amplified via a speaker 32. To assists the surgical team, the volume on the receiver system can be adjusted via conventional adjusting means 34.

Enabling the laparoscopic instrument 12 to adequately and successfully detect sound, a conventional radio frequency IC chip is utilized. Coupled to the radio frequency IC chip is the microphone. The purpose of the microphone is to sense the sounds for transmission from the laparoscopic instrument to the receiver system.

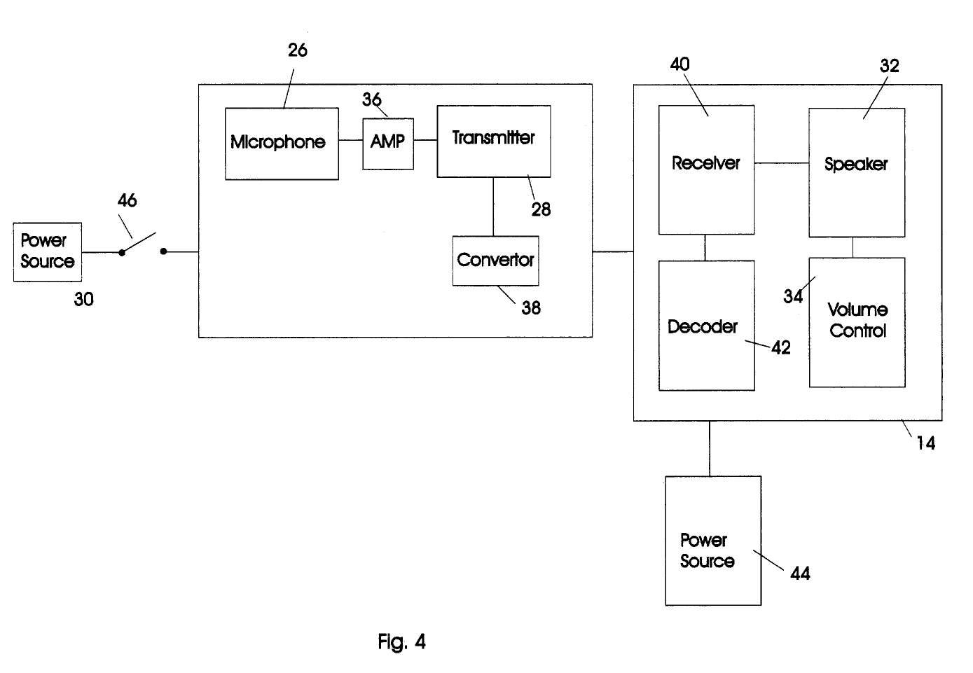

As seen in FIG. 3 and in the block diagram, illustrated in FIG. 4, sound sensed by the microphone 26 is converted to signals, which are amplified via the amplifier 36, and used to modulate a carrier wave that is generated by the transmitter 28. This modulated carrier is also amplified and then applied to a converter 38 for enabling the electrical signals to be converted to electromagnetic waves for transmission.

These electromagnetic waves are sent to the receiver system 14 via a receiver 40 and are intercepted and converted back to electrical signals via a decoder 42. These electrical signals are fed to the receiver 40 and separated from the carrier wave. Once separated, they are fed to the speaker 32, where they are converted into sound waves. The sound can be adjusted via conventional volume control means 34. The receiving system is powered via a power source 44.

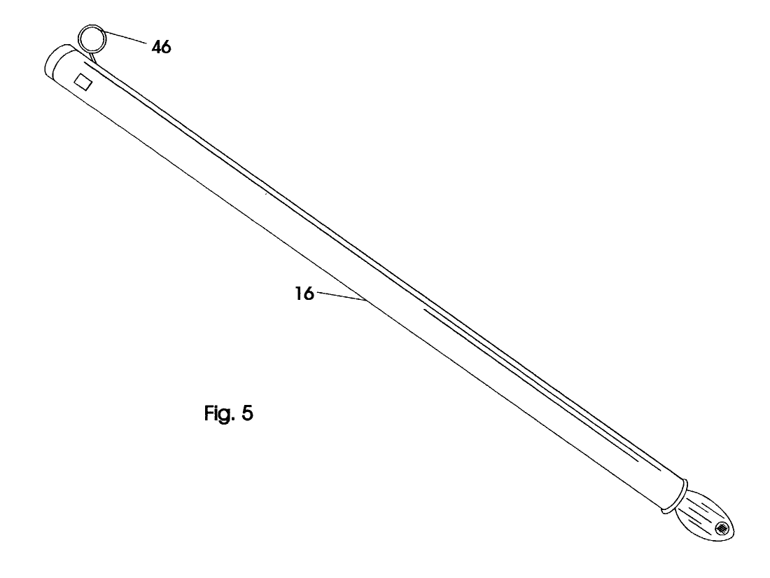

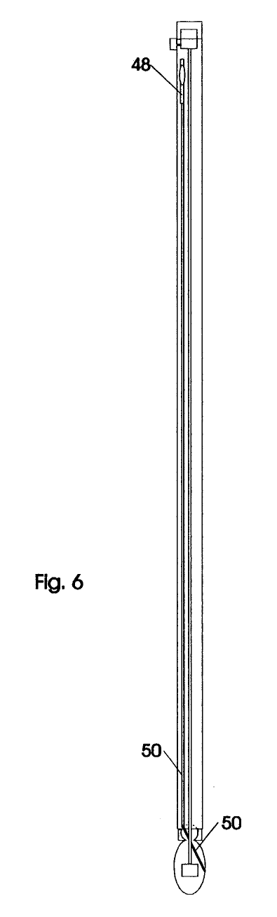

The instrument of the present invention can be altered so as to enhance the final product by providing a tip that can be moved as deemed necessary by the surgeon. This alternation of the surgical instrument is illustrated in FIGS. 5 and 6. As seen in these drawings, a movement device 46 having opposite ends is provided. The first end of the movement device extends exteriorly from the hollow tubular body 16. As seen in the drawings, the first end is structured as a handle so as to provide for the first end to receive the finger of the user and allow the user to manipulate the tip. This first end extends out from the interior of the hollow tubular body 16 through a channel 48. The second end is attached to the interior of the tip. The tip is secured to the second end of the hollow tubular body via conventional ball and socket means. This provides for the tip to be able to rotate freely about the second end of the hollow tubular body. Rigid link arms provide for the first end of the movement device to be coupled to the second end of the movement device. Thus, when moving the handle linearly within the channel will force the ball end of the tip to rotate about the second end of the hollow tubular member. This will move the tip as desired by the user.

Allowing movement of the tip will enable the surgeon to aim the instrument in any direction so as to sense the presence of any arterial Vessel structures, thereby alerting the surgeon performing the operation.

While the invention has been particularly shown and described be understood by those skilled in the art that various changes in form and detail may be made without departing from with reference to an embodiment thereof, it will the spirit and scope of the invention.

I claim:

1. A sound detection device useful in the performance of laparoscopic procedures on a human body, said device comprising:
a. an elongated hollow tube having a first and a second end thereof, said tube being formed of dimensions capable of passage through standard sized ports in said body which are used during said laparoscopic procedures;
b. a sound detector disposed in said first end of said tube for detecting sounds produced by anatomic vessels within said body; and,
c. a transmitter disposed in said tube and having an input coupled to an output of said sound detector for transmitting signals indicative of said sounds produced by said anatomic vessels.

2. The device as in claim 1 further including a receiver external to said tube and being tuned for receiving signals transmitted by said transmitter.

3. The device as in claim 2 further including an amplifier having an input coupled to an output of said receiver for amplifying signals received from said receiver.

4. The device as in claim 3 further including a speaker coupled to an output of said amplifier for converting said signals to sound waves, whereby a physician performing said laparoscopic procedure and listening to said sound waves may decipher anatomic vessels from other organs within said body.

5. The device a in claim 4 further including a volume control for adjusting the volume of sound from said speaker.

6. The device as in claim 1 further including a source of electrical energy.

7. A sound detection device useful in the performance of laparoscopic procedures on a body, said device comprising:
a. an elongated hollow tube having a first and a second end thereof, said tube being formed of dimensions capable of passage through standard sized ports in said body which are used during said laparoscopic procedures; and,
b. a sound detector disposed in said first end of said tube for detecting sounds produced by anatomic vessels within said body.

8. The device as in claim 7 further including an amplifier having an input coupled to an output of said sound detector for amplifying signals received therefrom.

9. The device as in claim 8 further including a speaker coupled to an output of said amplifier for converting converting said signals to sound waves, whereby a physician performing said laparoscopic procedure and listening to said sound waves may decipher anatomic vessels from other organs within said body.

10. The device a in claim 9 further including a volume control for adjusting the volume of sound from said speaker.

11. The device as in claim 7 further including a source of electrical energy.

12. A sound detection device useful in the performance of laparoscopic procedures on a body, said device comprising:
a. an elongated hollow tube having a first and a second end thereof, said tube being formed of dimensions capable of passage through standard sized ports in said body which are used during said laparoscopic procedures;
b. a sound detector disposed in said first end of said tube for detecting sounds produced by anatomic vessels within said body;
c. a transmitter disposed in said tube and having an input coupled to an output of said sound detector for transmitting signals indicative of said sounds produced by said anatomic vessels;
d. a receiver external to said tube and being tuned for receiving signals transmitted by said transmitter;
e. an amplifier having an input coupled to an output of said receiver for amplifying signals received from said receiver; and,
f. a speaker coupled to an output of said amplifier for converting said signals to sound waves, whereby a physician performing said laparoscopic procedure and listening to said sound waves may decipher anatomic vessels from other organs within said body.

13. The device a in claim 9 wherein said amplifier further includes a volume control for adjusting the volume of sound from said speaker.

14. The device as in claim 12 further including a source of electrical energy.
